(19) United States
(12) Patent Application Publication
(10) Pub. No.: US 2008/0318247 A1
(43) Pub. Date: Dec. 25, 2008

(54) Biomarker for cardiac transplant rejection

(75) Inventors: Chang Chang, Philadelphia, PA (US); Howard J. Eisen, Wynnewood, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Drexel University, Philadelphia, PA (US); Philadelphia Health & Education Corporation, d/b/a Drexel University College of Medicine, Philadelphia, PA (US)

(21) Appl. No.: 12/080,285

(22) Filed: Apr. 2, 2008

Related U.S. Application Data
(60) Provisional application No. 60/921,928, filed on Apr. 4, 2007.

(51) Int. Cl. G01N 33/53; C12Q 1/02

(52) U.S. Cl. 435/7.1; 435/29

(57) ABSTRACT

The invention provides a method of diagnosing a disease or disorder featuring an abnormal level of a ring-containing molecule in a tissue. In one embodiment, a method of diagnosing organ transplant rejection is provided.

### CROSS-REFERENCE TO RELATED APPLICATIONS

This application claims the benefit pursuant to 35 U.S.C. §119(e) of U.S. Provisional Application No. 60/921,928, filed Apr. 4, 2007, which is hereby incorporated by reference in its entirety herein.

### BACKGROUND OF THE INVENTION

Organ transplantation is the preferred clinical approach to treat end-stage organ failure or complications arising from diseases of specific organs. However, transplant patients face a lifetime of immunosuppressive therapy and the risk of losing the new organ due to rejection. Transplant rejection occurs when the immune system of the recipient of a transplant attacks the transplanted organ or tissue. This immune response occurs because a normal healthy human immune system can distinguish foreign tissues and attempts to destroy them. Rejection is an adaptive immune response and is mediated through both T cell mediated and humoral immune (antibodies) mechanisms. Constant vigilance is required to monitor the immune response to the grafted organ. Acute rejection occurs in the first 6 months after transplantation. Chronic rejection, occurring at least 6 months after transplantation, is very difficult to diagnose clinically and usually presents as a gradual vasculopathy of grafted vessels.

Acute rejection is generally acknowledged to be mediated by T cell responses to proteins from the donor organ which differ from those found in the recipient. Unlike antibody-mediated hyperacute rejection, development of T cell responses first occurs several days after a transplant if the patient is not taking immunosuppressant drugs. Since the development of powerful immunosuppressive drugs, such as cyclosporin, tacrolimus and rapamycin, the incidence of acute rejection has been greatly decreased. However, organ transplant recipients can develop acute rejection episodes months to years after transplantation. Acute rejection episodes can destroy the transplant if it is not recognized and treated appropriately. Episodes occur in around 60-75% of first kidney transplants, and 50 to 60% of liver transplants. A single episode is not a cause for concern if recognised and treated promptly and rarely leads to organ failure, but recurrent episodes are associated with chronic rejection of grafts.

Chronic rejection occurs months to years following transplantation. It is characterized by graft arterial occlusions, which results from the proliferation of smooth muscle cells and production of collagen by fibroblasts. This process, termed accelerated or graft arteriosclerosis, results in fibrosis which can cause ischemia and cell death. These fibrous lesions occur without evidence of an overt cause (such as vascular injury or infection), although it is hypothesized that chronic rejection is really the result of continued prolonged multiple acute rejections.

As with other end-stage diseases, the standard treatment for end-stage cardiac diseases is heart transplantation. The efficacy of heart transplantation is limited by allograft rejection (Eisen, H. J. et al., 2003, New England J. Med. 349: 847-858). Physicians typically monitor patients for organ rejection following a heart transplant by performing frequent endomyocardial biopsies for the first year. Endomyocardial biopsies are invasive procedures that involve threading a catheter through the internal jugular vein to the heart's right ventricle and snipping out several, typically four, tiny pieces of tissue. A pathologist then tests the tissue to identify the presence of immune cells (such as macrophages) as well as other pathological changes in the transplanted heart tissue that indicate the graft is being rejected by the body's immune system. Thus, endomyocardial biopsy has been the gold standard for rejection surveillance, where histopathology is used to classify the severity of the allograft rejection from Grade 0 (no rejection) to Grade 4 (severe) (Stewart et al., 2005, J Heart Lung Transplant 24:1710-1720; Baumgartner, Heart and Lung Transplantation, Edn. 2nd., W.B. Saunders, Philadelphia, Pa., 2002). However, heart biopsy is invasive, subject to inter-observer variability, and causes morbidity (0.5-1.5%) (Deng et al., 2006, Am J Transplant 6:150-160).

Thus, there is a need in the art for improved method for monitoring transplant rejection. The present invention addresses this need.

### BRIEF SUMMARY OF THE INVENTION

The invention provides a method of diagnosing a disease or disorder featuring an abnormal level of a ring-containing molecule in a tissue of a mammal. The method comprises assessing the level of the ring-containing molecule in the tissue, wherein a change in the level of the molecule in the tissue compared to a reference level of the molecule is indicative of the disease or disorder. In some embodiments, the ring-containing molecule contains a ring selected from the group consisting of a purine ring, a pyrimidine ring, an indole ring, an imidazole ring and a pyrrolidine ring. In some embodiments, the ring-containing molecule is selected from the group consisting of adenine, guanine, cytosine, thymidine, uracil, inosine, xanthine, tryptophan, tyrosine, phenylalanine, histidine, serotonin, proline and naturally-occurring derivatives thereof.

The invention also provides a method of diagnosing organ transplant rejection in a mammal. The method comprises assessing the level of serotonin in a transplanted organ or a tissue sample obtained from the mammal into whom the organ has been transplanted, wherein an elevated level of serotonin in the transplanted organ or the tissue sample compared to a reference level of serotonin is indicative of transplant rejection. In some embodiments, the organ transplant comprises an organ selected from the group consisting of heart, heart valves, lung, kidney, liver, cornea, pancreas, heart, intestine, tendons, skin, neural tissues and combinations thereof. In a preferred embodiment, the organ transplant comprises a heart transplant. In another preferred embodiment, the organ transplant comprises a kidney transplant.

In the method of a method of diagnosing a disease or disorder featuring an abnormal level of a ring-containing molecule and in the method of diagnosing organ transplant rejection, the mammal is preferably a human. Raman spectroscopy or immunoassay may be used in either method for assessment.

In some embodiments of the methods, assessing the level of serotonin comprises using Raman spectroscopy. Assessing the level of serotonin may comprise using Raman spectroscopy in vivo or in vitro. In some embodiments, assessing the level of serotonin comprises assessment of one or more Raman peaks selected from the group consisting of about 678 cm⁻¹, about 758 cm⁻¹, about 820-860 cm⁻¹ and about 938 cm⁻¹. In preferred embodiments, assessing the level of serotonin comprises assessment of the about 758 cm⁻¹ Raman peak.

In some embodiments, assessing the level of serotonin comprises obtaining Raman spectra at multiple positions in the transplanted organ or tissue sample obtained therefrom. In some embodiments, assessing the level of serotonin comprises calculating a ratio of a Raman peak that is indicative of serotonin to a reference Raman peak that is not affected by serotonin. In one aspect, the Raman peak that is indicative of serotonin is about 758 cm⁻¹ and the reference Raman peak is about 718 cm⁻¹.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A, 1B and 1C are a series of images depicting Raman spectra of a biopsy and microscopic images of the biopsy. FIG. 1A depicts spatially-resolved Raman spectra of an endomyocardial biopsy. Spectra 1, 2 and 10 obtained from sites of normal myocardium. Spectra 3-9 obtained from sites of cardiac fibrosis. The wavelengths (±3 cm⁻¹) of notable peaks are indicated vertically above the corresponding peak. The number at the right of each spectrum corresponds to the mapping position indicated in FIG. 1B. FIG. 1B is a microscopic image of the biopsy seen with a 10× objective. The locations examined by Raman spectroscopy are marked by 1 to 10. FIG. 1C is a microscopic image of the adjacent endomyocardial biopsy section stained with haematoxylin and eosin (H&E). The section shown in FIG. 1C was adjacent to the section in FIG. 1B.

FIG. 2 depicts the Raman spectra of serotonin (5-HT) dissolved in phosphate buffered saline (PBS) solution; normal myocardium (spectra of map positions 1, 2 and 8 in FIG. 1B averaged together); and cardiac fibrosis (spectra of map positions 3-7, 9 and 10 in FIG. 1B averaged together).

FIGS. 3A-3D are a series of images depicting heart biopsies samples and graphs of Raman spectra obtained at randomly-selected positions in heart biopsy samples. FIGS. 3A and 3B are microscopic images of a Grade 1 and a Grade 2 biopsy, respectively. Markings in the microscopic images designate the randomly-selected positions where Raman spectra were obtained. FIGS. 3C and 3D depict the corresponding Raman spectra. The spectra were obtained without prior knowledge of the rejection grading of the biopsies. In FIG. 3D, spectra 8-12 exhibit a Raman band at 678 cm⁻¹. The numbers on the right correspond to the mapping positions indicated in FIGS. 3A and 3B, respectively.

FIG. 4 depicts averaged Raman spectra from six Grade 0 biopsies (spectra 1) and four Grade 2 biopsies (spectra 2). The first spectrum shows the average of 66 Raman spectra obtained from the Grade 0 biopsies. The 16 Raman spectra obtained from Grade 2 biopsies that do not possess the 678 cm⁻¹ peak are averaged into the second spectrum. The third spectrum is the average of the 17 Raman spectra which show the 678 cm⁻¹ peak. The onset at 678 cm⁻¹ and strengthening at 758 cm⁻¹ are clearly observable.

FIGS. 5A and 5B are a series of images of a heart biopsy sample stained for serotonin and for collagen. FIG. 5A is a heart biopsy section immunohistochemically stained to detect serotonin. FIG. 5B is a heart biopsy section adjacent to the one depicted in FIG. 5A and which is stained with Masson's TriChrom. Arrows denote regions of collagen, indicative of fibrosis.

### DETAILED DESCRIPTION OF THE INVENTION

The present invention springs in part from the discovery that serotonin is the major differentiating molecular marker that accumulates at the site of fibrosis in a transplanted graft. Specifically, the level of serotonin in a transplanted graft correlates with histopathological indicators of transplant rejection. Accordingly, the present invention provides a method of diagnosing transplant rejection by assessing the level of serotonin in the transplant graft. The invention also springs in part from the discovery that Raman spectroscopy can be used to detect in tissues a wide array of molecules that contain ring structures. Thus, in a preferred embodiment, the level of serotonin is assessed using Raman spectroscopy.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, an “immunoassay” refers to any binding assay that uses an antibody capable of binding specifically to a target molecule to detect and quantify the target molecule.

“Graft” refers to any free (unattached) cell, tissue or organ for transplantation.

“Allograft” refers to a transplanted cell, tissue or organ derived from a different animal of the same species.

“Xenograft” refers to a transplanted cell, tissue or organ derived from an animal of a different species.

“Specifically bind” as used herein refers to the higher affinity of a binding molecule for a target molecule compared to the binding molecule's affinity for non-target molecules. A binding molecule that specifically binds a target molecule does not substantially recognize or bind non-target molecules.

“Transplant rejection” as used herein refers to one or more of a variety of biological processes that contribute to the progressive deterioration of biological function and physical integrity of an allo- or xenograft in the graft recipient. Such processes include immunological rejection as well as chronic transplant vasculopathy that leads to cardiac fibrosis. The term therefore embraces both acute rejection and chronic rejection.

As used herein, “diagnose” refers to detecting and identifying a disease in a subject. The term also encompasses assessing or evaluating the disease status (progression, regression, stabilization, response to treatment, etc.) in a patient known to have the disease.

The terms “assessing” and “evaluating” are used interchangeably to refer to any form of measurement, and includes determining is an element is present or not. Assessing may be relative or absolute.

As used herein, a “reference level” refers to the level of a molecule present in one or more samples of a tissue not afflicted with a disease or disorder that features abnormal level of the molecule.

As used herein with respect to graft transplantation, “reference serotonin level” refers to the level of serotonin present in one or more samples of a non-rejected graft. The skilled artisan is familiar with establishing such reference data. Reference data may be obtained from a single sample or from a multitude of samples, such as a collection of biopsy samples from non-rejected hearts. In one embodiment, the assessment of serotonin elevation is made by comparison to one or more reference levels of serotonin in regions of the transplanted graft that do not manifest clinical signs of rejection. For instance, the assessment of serotonin elevation may be made by calculating a ratio of a Raman peak that is indicative of serotonin to a reference Raman peak that is not affected by serotonin in the transplanted graft. Reference data may be specific for particular populations. For instance, reference data may be stratified based on gender and age range or on exposure to immunosuppression.

As used herein, a “ring structure” refers to a five- or six-membered ring. A ring structure may be monocyclic or heterocyclic and may be fused to other rings. Representative examples of ring structures are purines, imidazoles, pyrimidines, pyrrolidines and indoles.

As used herein, a “ring-containing molecule” is a molecule that contains a ring structure.

“Naturally-occurring” as applied to an object refers to the fact that the object can be found in nature. For example, a ring-containing molecule that is present in an organism (including viruses) that can be isolated from a source in nature and which has not been intentionally modified by man is naturally-occurring.

It is understood that any and all whole or partial integers between any ranges set forth herein are included herein.

### DESCRIPTION

The invention provides methods of detecting the level of a molecule comprising a ring structure in a tissue to diagnose a disorder or disease featuring a change in the concentration of the molecule in a tissue or organ. In one embodiment, the level of serotonin is detected in a tissue graft to diagnose transplant rejection.

The methods of the invention may be used in any mammal, including a human. Non-human animals subject to diagnosis include, for example, primates, mice, rats, cattle, sheep, goats, horses, canines, felines and the like. The methods are preferably used in a human.

The invention is based in part on the discovery that Raman spectroscopy can specifically detect molecules comprising a ring structure in a biological tissue. Specifically, it has been discovered that Raman spectroscopy can be used to detect serotonin in tissue, In addition, it has been discovered that an elevated level of serotonin in a transplanted graft correlates with an increase in (signs of/risk of) transplant rejection. Thus, the invention provides methods of diagnosing a disorder or disease featuring a change in the level of a ring-containing molecule in tissue by means of Raman spectroscopy. Preferably, the ring-containing molecule is naturally occurring.

Naturally-occurring molecules comprising a ring structure that can be detected by Raman spectroscopy include, but are not limited to, nucleotides and oligomers thereof, certain amino acids and derivatives thereof. Specific molecules include, but are not limited to adenine and guanine, cytosine, thymidine, uracil, inosine, xanthine, tryptophan, tyrosine, phenylalanine, histidine, and proline and naturally-occurring derivatives thereof. Non-limiting examples of naturally-occurring derivatives include monoamines including serotonin (5-hydroxytryptamine), melatonin, catecholamines (tyrosine derivatives such as dopamine (4-(2-aminoethyl)benzene-1,2-diol), norepinephrine and epinephrine), 5-hydroxytryptophan, histamine, trace amines, thryoid hormones (e.g. triiodothyronine and thryoxine), thyronamines and the like.

Numerous diseases and disorders that involve an increase or decrease, compared to normal or non-diseased tissue, of the level of a ring-containing molecule in a tissue are known in the art. Representative examples include phenylketonuria and orthostatic hypertension. Phenylketonuria is caused by a deficiency of phenylalanine hydroxylase and features an abnormal level of phenylalanine in the brain and plasma. Assessing phenylalanine levels in the brain is thought to be more accurate than plasma phenylalanine, however, brain levels are difficult to measure. Advantageously, the method of the invention overcomes such difficulty by means of in vivo Raman spectroscopy. Orthostatic hypertension is caused by a deficiency in dopamine β-hydroxylase and features an abnormal elevation of dopamine in cerebrospinal fluid (CSF). Preeclampsia is thought to involve reduced placental indoleamine 2,3-dioxygenase activity, which thus affects the amount of tryptophan present. Accordingly, assessing the tryptophan level or the ratio of kynurenine to tryptophan in the placenta may be useful for diagnosing preeclampsia. Other diseases or disorders featuring an abnormal level of a ring-containing molecule include pheochromocytoma, Von Hippel-Lindau, gout, which features deposition of monosodium urate crystals on the articular cartilage of joints and in the particular tissues like tendons, xanthinuria, Lesch-Nyhan syndrome and severe combined immunodeficiency disease (SCID). However, the invention should not be construed as being limited to the disease discussed herein. The skilled artisan is familiar with other diseases and disorders that may be diagnosed or monitored using the methods of the invention. In addition, diseases or disorders featuring abnormal levels of a ring-containing molecule, which are at present unknown, once known, may also be diagnosable using the methods of the invention.

The invention is also based in part on the discovery that serotonin is elevated in a transplanted graft undergoing transplant rejection. Therefore, in a preferred embodiment, the method is used to diagnose transplant rejection by determining if the level of serotonin in the transplanted graft is elevated. To determine whether serotonin is elevated, the level of serotonin is measured in a transplanted graft, or a sample thereof, and is compared to a reference serotonin level or range of levels that corresponds to tissue of the same type as the graft that is not undergoing rejection. In one embodiment, a single serotonin level is measured in a transplanted graft and compared with a reference serotonin level. In another embodiment, serotonin levels are determined at multiple different positions in a transplanted graft to generate an array of serotonin levels that are compared with reference serotonin levels. Similarly, the level of any ring-containing molecule can be detected in a tissue, preferably using Raman spectroscopy, and is compared to a reference level for that molecule in order to diagnose a disorder or disease related to an abnormal level of the molecule. Optionally, measuring the level of serotonin in the transplanted graft comprises the use of a catheter-based fiber optics probe.

The methods of the invention may be used to diagnose any graft transplant for rejection. Grafts of interest include: kidney, lung, cornea, liver, pancreas, pancreas islets, heart, heart valves, stomach, large intestine, small intestine, muscle, bladder, tendons, neural tissues and skin. Preferably, the method of the invention is used to evaluate rejection in transplanted kidney, lung, liver, pancreas, heart, bladder and combinations thereof. More preferably, the methods of the invention are used to diagnose rejection in a transplanted heart. In another embodiment, the methods of the invention are used to diagnose rejection in a transplanted kidney. The methods of the invention may be used for both allografts or xenografts transplants.

The method of diagnosing transplant rejection can replace standard, prior art methods of diagnosing organ rejection, such as histopathological evaluation of tissue biopsies, or can supplement them. Advantageously, the method of the invention provides a more objective evaluation of transplant rejection than current methods of transplant rejection monitoring. Furthermore, when performed in vivo, the method of the invention is less invasive, as it does not need to incise the graft, and less likely to cause or contribute to morbidity of the subject animal. Similar advantages are contemplated in applying the inventive method to diagnosing a disorder or disease related to an abnormal level of other ring-containing molecules. In the methods of the invention, the measurement of a ring-containing molecule level is accomplished through any means known in the art. Non-limiting exemplary methods of measuring ring-containing molecule levels include Raman spectroscopy of tissue samples, in vivo Raman spectroscopy and immunostaining of tissue samples.

Tissue samples from transplanted grafts are obtained by conventional biopsy methods in the art. Tissue preparation or fixation is desirable for the preservation of cell morphology and tissue architecture. Inappropriate or prolonged fixation may significantly diminish the antibody binding capability of the tissue. Many antigens can be successfully detected in formalin-fixed paraffin-embedded tissue sections. However, some antigens will not survive even moderate amounts of aldehyde fixation. Under these considerations, tissues should be rapidly fresh frozen in liquid nitrogen and cut with a cryostat. The disadvantages of frozen sections include poor morphology, poor resolution at higher magnifications, difficulty in cutting over paraffin sections, and the need for frozen storage. Alternatively, vibratome sections do not require the tissue to be processed through organic solvents or high heat, which can destroy the tissue's antigenicity, or be disrupted by freeze thawing. The disadvantage of vibratome sections is that the sectioning process is slow and difficult with soft and poorly fixed tissues, and that chatter marks or vibratome lines are often apparent in the sections. Preferably, fresh or frozen tissue samples are used for ex vivo Raman spectroscopy.

Raman spectroscopy is a well-established analytical tool for obtaining compound-specific information for chemical analysis. See, for instance, Smith et al., Modern Raman Spectroscopy: A Practical Approach, J Wiley, Hoboken, N.J., 2005. It is based on an optical phenomenon, Raman scattering, where photons are inelastically scattered by a molecule. The changes in the vibrational states of the molecule are accompanied by the frequency shifts in the scattered photons. By analyzing the spectral distribution of such photons, Raman spectroscopy provides the characteristic vibrational information about the chemical bonds of the sample under study. Molecularly and chemically specific information can therefore be obtained using Raman spectroscopy. Raman spectroscopy has been used to analyze biological tissues. See, for instance, U.S. Patent Application Publication No. 20060281068 and references therein. Thus, there is substantial guidance in the art on the application of Raman spectroscopy to biological tissues. Advantageously, Raman spectroscopy does not require staining and labeling of a tissue.

An exemplary method of using Raman spectroscopy to detect a ring-containing molecule, e.g., serotonin, in tissue sections is described in the Examples herein. Any radiation wavelength suitable for Raman spectroscopy may be used in the invention. Generally, wavelengths from about 700 nm to about 1000 μm are useful. Preferably, the irradiation laser used in the practice of the invention is at least about 780 nm and more preferably, at least about 830 nm. For instance, Raman peaks (±3 cm⁻¹) that are indicative of serotonin levels include about 678 cm⁻¹, about 758 cm⁻¹, about 820-860 cm⁻¹ and about 938 cm⁻¹. In one embodiment, elevation of serotonin level in a tissue is assessed based on one of these Raman peaks. In a preferred embodiment, the about 678 cm⁻¹ peak is used to assess serotonin level. In another preferred embodiment, the about 758 cm⁻¹ peak is used. In another embodiment, elevation of serotonin is assessed based on two or more Raman peaks. In some embodiments, the existence of 678 cm⁻¹ Raman peak corroborates the elevated level of serotonin. For instance, in one embodiment, the about 678 cm⁻¹ and the about 758 cm⁻¹ peaks are used to assess serotonin level. As demonstrated herein for cardiac transplant tissue, the onset of the peak at about 678 cm⁻¹, is consistently accompanied by enhanced Raman signals at about 758 cm⁻¹, and about 938 cm⁻¹. Therefore, in yet another embodiment, serotonin level is assessed based on Raman peaks at about 678 cm⁻¹, about 758 cm⁻¹ and about 938 cm⁻¹. In yet another embodiment, serotonin level is assessed based on a range of peaks from about 600 cm⁻¹ to about 1000 cm⁻¹. In any of the embodiments, the intensity of a diagnostic peak may be normalized, for instance, by comparison to the intensity of a Raman peak not known to be affected by the level of serotonin. In one embodiment, the ratio of the intensity of the Raman peak at about 758 cm⁻¹ (I₇₅₈) to the intensity of the peak at about 718 cm⁻¹ (I₇₁₈) is used. In this embodiment, the reference level of the intensity ratio is 1. A scan whose I₇₅₈/I_{758 is} greater than 1 is deemed to reflect an elevated level of serotonin. The skilled artisan, armed with the present disclosure, is readily able to determine the Raman peaks that identify other ring-containing molecules, such as nucleotides and aromatic amino acids and thus are useful in the practice of the invention.

Raman spectroscopy's non-contact optical nature eliminates the need for tissue removal, enabling in vivo application of the technique. Frequent, noninvasive monitoring of transplanted grafts, as is the case using Raman spectroscopy, is advantageous because it allows physicians to better tailor immunosuppression drug regimens according to individual patient's needs, for instance, by prescribing lower doses to more stable patients or increasing doses for patients who exhibit early signs of rejection.

In vivo Raman spectroscopy generally uses low-background, small diameter, optical fiber probes (Motz et al., 2004, Appl Opt. 43:542-554; Motz et al., 2005, J Biomed Opt 10:031113). The Raman probe is positioned in near proximity or touching the transplant graft and spectra are detected. Raman probes may be used in the bloodstream and in endoscopic procedures, including, but not limited to, colonoscopy, esophagogastroduodenoscopy, laproscopy, proctosigmoidoscopy, bronchoscopy, cystoscopy, arthroscopy, thoracoscopy and mediastinoscopy.

The level of a ring-containing molecule such as serotonin may also be measured using an immunoassay to detect serotonin in a tissue section. Immunoassays useful in the present invention include, for example, immunohistochemistry assays, immunocytochemistry assays, ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor Laboratory Press, Cold Spring Harbor, N.Y.; Harlow et al., 1999, Using Antibodies: A Laboratory Manual, Cold Spring Harbor Laboratory Press, Cold Spring Harbor, N.Y. Tissue sections used for immunostaining may or may not be fixed. Preferably, the tissue section is fixed. Levin et al. (2004, J Biochem Biophys Methods 58:85-96), incorporated herein by reference, disclose a non-limiting method of immunohistochemical detection of serotonin in tissue samples.

Antibodies that specifically detect serotonin or another ring-containing molecule may be obtained using techniques known in the art. Preferably, the antibody specifically binds human ring-containing molecules, such as human serotonin. Antibodies that specifically bind serotonin are commercially available. Antibody vendors include Chemicon, AbD Serotec, Invitrogen, Dako and Sigma-Aldrich. Alternatively, anti-serotonin antibodies useful in practicing the invention can be generated by conventional methods known to the skilled artisan.

The generation of polyclonal antibodies is accomplished by inoculating the desired animal with the antigen and isolating antibodies which specifically bind the antigen therefrom.

Monoclonal antibodies directed against serotonin, or any other ring-containing molecule, may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Quantities of serotonin may be synthesized using chemical synthesis technology or in vitro enzymatic synthesis from tryptophan using tryptophan hydroxylase and amino acid decarboxylase. Alternatively, serotonin may be purified from a biological source that endogenously comprises serotonin, or from a biological source recombinantly-engineered to produce or over-produce serotonin. Monoclonal antibodies directed against serotonin are generated from mice immunized with serotonin using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody may be engineered such that the protein is displayed on the surface thereof in such a manner that it is available for binding to the antigen against which the antibody is directed. Thus, when bacteriophage which express a specific antibody are incubated in the presence of the antigen, for instance, antigen immobilized on a resin or surface, the bacteriophage will bind to the antigen. Bacteriophage which do not express the antibody will not bind to the antigen. Such panning techniques are well known in the art and are described for example, in Wright et al., (supra).

Processes, such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, phage which encode single chain antibodies (scFv/phage antibody libraries) are also useful in preparing Fab molecules useful in the invention. Fab molecules comprise the entire Ig light chain, that is, they comprise both the variable and constant region of the light chain, but include only the variable region and first constant region domain (CHI) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA. Synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105) may also be used to prepare an antibody useful in the practice of the invention.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

The materials and methods used in the following Experimental Examples are now described.

Biopsy sample: Surveillance endomyocardial biopsies were obtained from heart transplant patients meeting the selection criteria approved by the Institutional Review Board of Drexel University College of Medicine for this study. Informed consent was obtained from each patient. A total of eleven endomyocardial biopsies were obtained. The ten biopsies used in Experimental Example 2 were from 10 different patients. The biopsy used in Experimental Example 1 was from one the 10 patients. Biopsy samples were quick/snap-frozen in liquid nitrogen and stored at −80° C. until use. Immediately prior to use, samples were passively thawed at room temperature and placed between two calcium fluoride slides for Raman spectroscopy. The edges of the calcium fluoride slides were concealed by petrolatum to prevent the biopsy samples from drying out.

Pathology: Biopsy grade was ascertained using standard criteria. The severity of cardiac allograft rejection is classified into 4 categories: from Grade 0 (no rejection) to Grade 4 (severe rejection). Grade 1 rejection is characterized by white blood cell infiltration with no myocyte damages. From Grade 2 rejection and up, the infiltration of white blood cells, focal or diffuse, are seen to be increasingly widespread and associated with myocyte damages (Billingham et al., 1990, J Heart Transplant. 9:587-93 and Cary, 1998, Heart 79:423-424). Although a biopsy may be assessed a Grade 1 or higher, it may still have regions that are normal looking.

H&E Staining: Hemotoxylin and eosin staining was performed using an commercially available machine.

Raman Spectra: The Raman spectroscopy experiments were conducted using a confocal Raman microscope (LabRAM 800HR, Horiba JobinYvon, Edison, N.J.). The intensity of the obtained Raman spectra was peak-normalized and offset vertically for display. No other data processing procedure was performed.

### Experimental Example 1

### Raman Spectroscopic Detection of Serotonin in Endomyocardial Biopsy

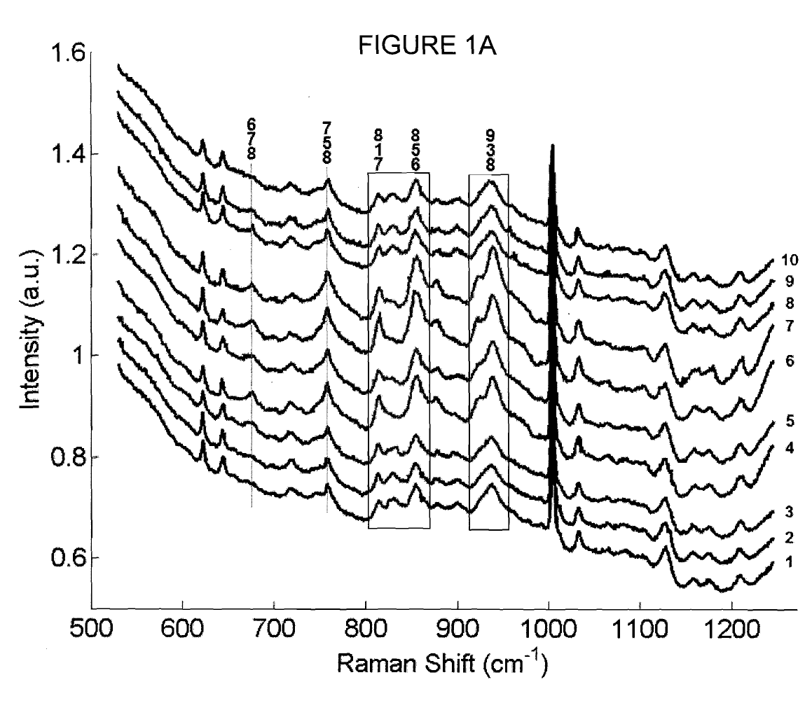

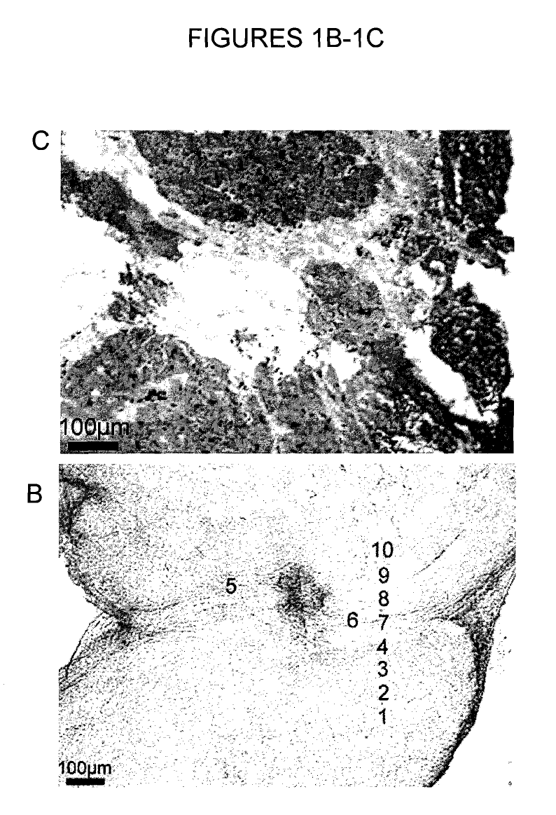

To assess whether Raman spectroscopy can detect serotonin ((5-hydroxytryptamine, 5-HT)) directly in a tissue biopsy, a grade 2 endomyocardial biopsy was cryosectioned and examined. One slice of the biopsy was subject to H&E staining, and the remaining bulk was examined using Raman spectroscopy. FIG. 1A shows a spatially-resolved Raman spectra of the endomyocardial biopsy.

The Raman spectra obtained from the area of fibrosis (positions 3-7, 9, 10 in FIG. 1B) were clearly distinguishable from those obtained from normal myocardium (positions 1, 2 and 8 in FIG. 1B). The four Raman bands of serotonin at 678 cm⁻¹, 758 cm⁻¹, 820-860 cm⁻¹ and 938 cm⁻¹ consistently emerged and intensified in regions of cardiac fibrosis. As shown in FIG. 1, the emergence of 678 cm⁻¹ Raman peak correlates reliably with fibrosis. In addition, the onset at 678 cm⁻¹ was consistently accompanied by enhanced Raman signals at 758 cm⁻¹ and 938 cm⁻¹. Changes in the 820 cm⁻¹-860 cm⁻¹ spectral region were also observed to be associated with the 678 cm⁻¹ band. It is noted that the Raman spectrum of collagen typically has two intense bands around 855 cm⁻¹ and 938 cm⁻¹. Therefore, the increased collagen concentration associated with fibrosis may contribute to some of the enhancements detected at these two spectral positions.

FIG. 1C shows the H&E stained adjacent cryosection that was adjacent to the section used for Raman spectroscopy. The H&E stained image of the adjacent cryosection clearly shows the horizontally-distributed cardiac fibrosis.

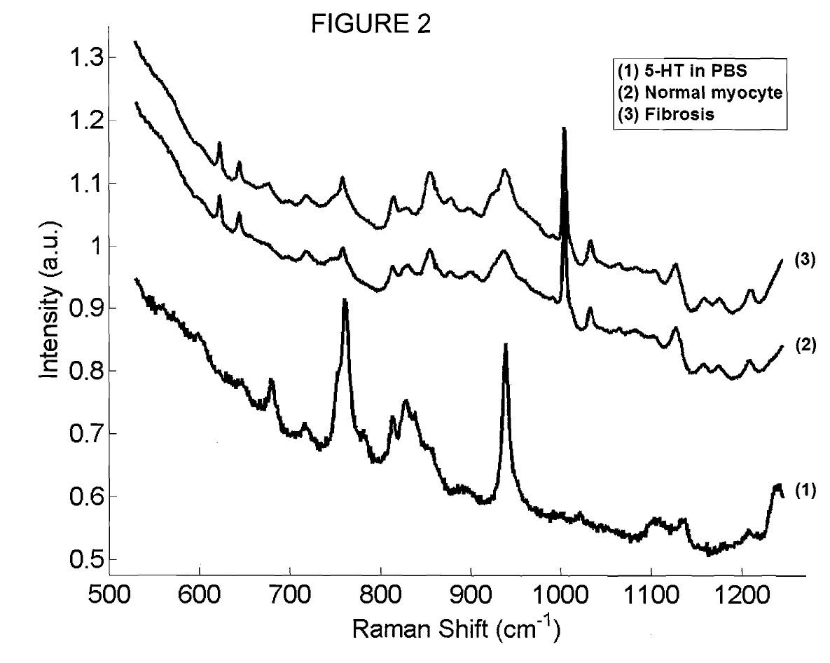

Raman spectrum of serotonin dissolved in phosphate buffered saline (PBS) solution is shown in FIG. 2. The spectrum of serotonin was compared with the averaged Raman spectra from normal myocardium (positions 1, 2, 8) and cardiac fibrosis (positions 3-7, 9, 10). The four characteristic Raman peaks of serotonin emerged in accordance with the distribution of the cardiac fibrosis. These data confirm that serotonin is the major differentiating molecular marker accumulated at the site of fibrosis.

This experimental sample therefore demonstrates that Raman spectroscopy can be used to distinguish regions of cardiac fibrosis in unstained heart biopsies by directly detecting the elevated levels of serotonin. The same detection principle can be readily applied to other disease processes.

### Experimental Example 2

### Raman Spectroscopy of Endomyocardial Biopsy

In order to simulate in vivo clinical environments, ten endomyocardial biopsies were examined with Raman spectroscopy only. Raman spectra were obtained at randomly selected positions within the heart biopsy samples without prior knowledge of their rejection grading. These data were then compared with the histopathological readings for the biopsies. Six endomyocardial biopsies of Grade 0 and four of Grade 2 are examined in this manner.

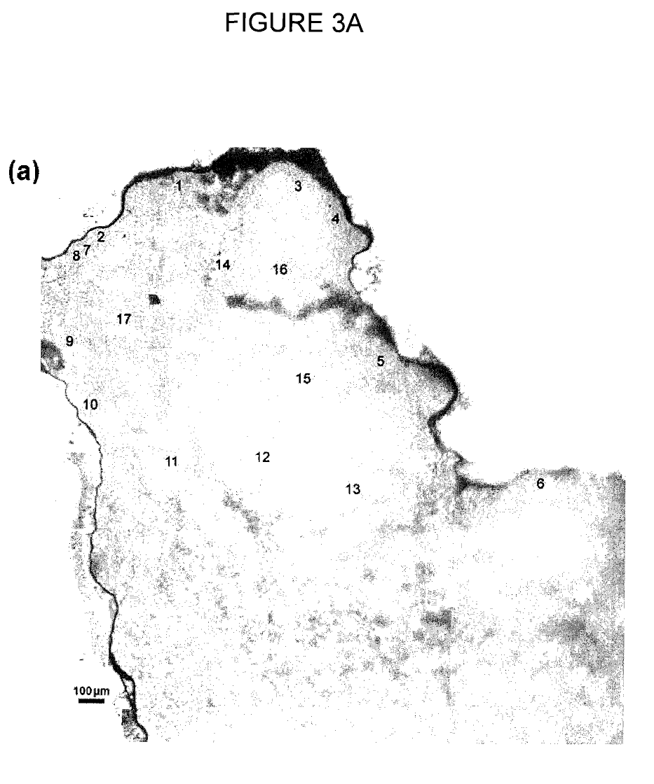

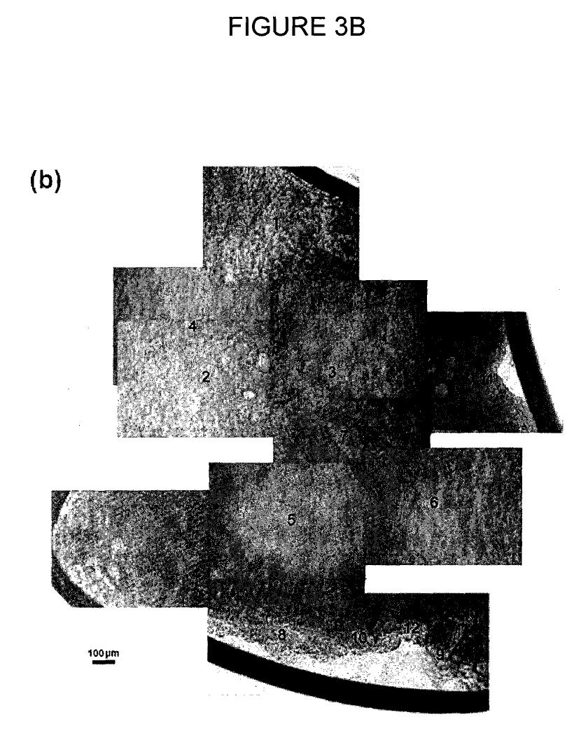

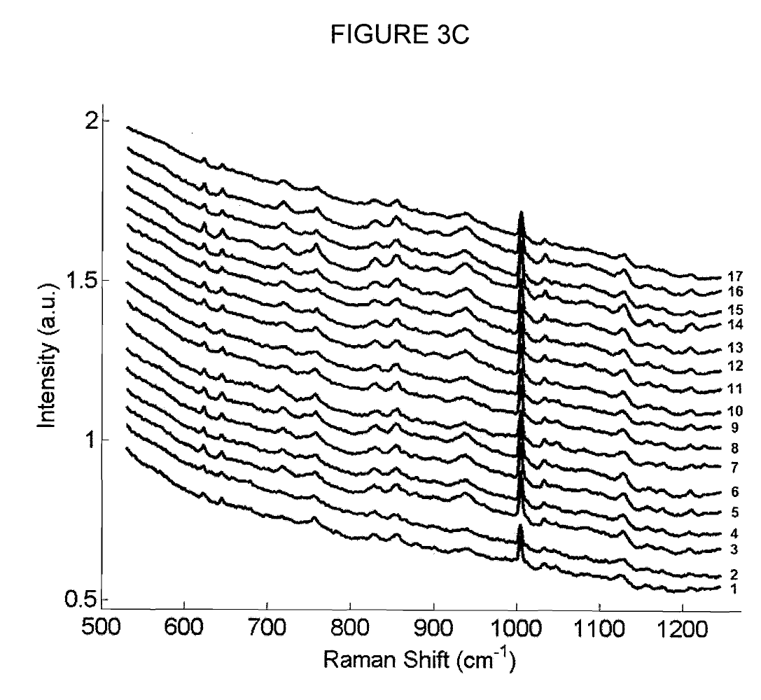

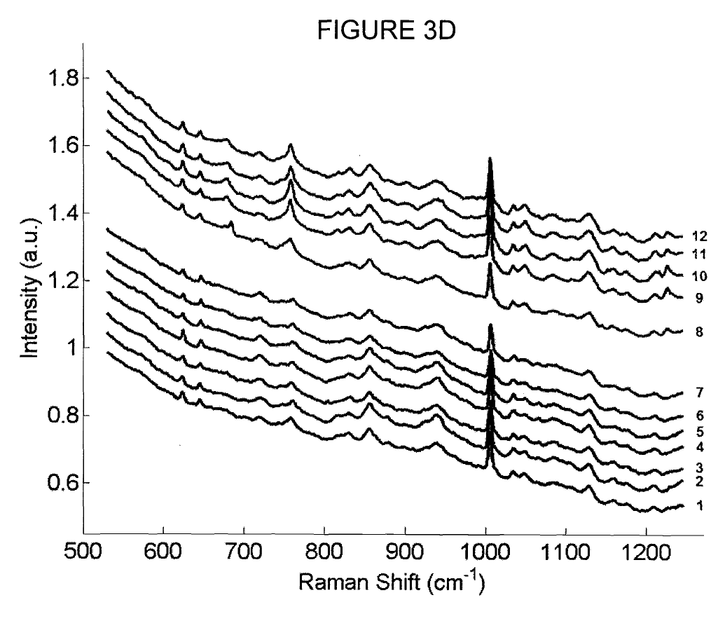

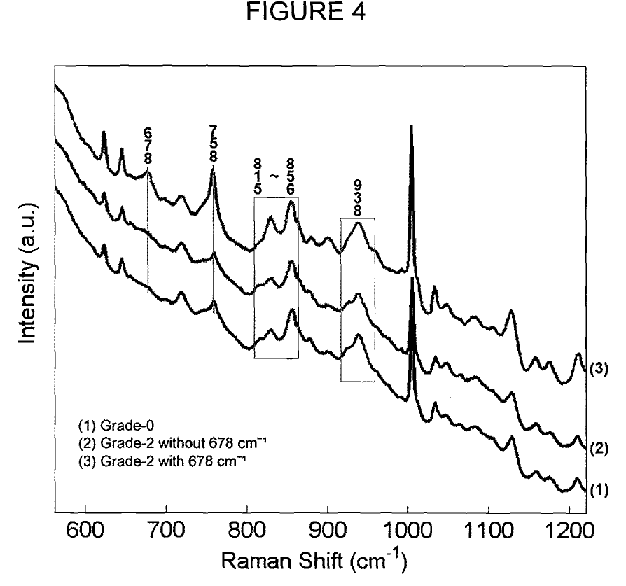

Four of the ten biopsies were found to have upticks at 678 cm⁻¹, indicative of an increased level of serotonin. These data were compared to the pathology readings for the ten biopsies. The upticks at 678 cm⁻¹ only occurred with Grade 2 biopsies. It is also observed that the onset of the 758 cm⁻¹ peak and associated changes in the spectra only occurred with Grade 2 biopsies. Representative Raman spectra are shown in FIG. 3. Raman bands of serotonin occurred only at some locations in the Grade 2 biopsy. Spectra obtained at other positions of normal myocardium on the Grade 2 biopsy are indistinguishable from those of the Grade 0 biopsy. This result indicates the consistency of Raman spectroscopy on normal myocardium. Averaged Raman spectra from the six Grade 0 and the four Grade 2 biopsies are shown in FIG. 4, again exhibiting the consistency of the Raman spectral detection.

Thus, the characteristic 678 cm⁻¹ Raman peak consistently emerged in Grade 2, but not in Grade 0 biopsies. In addition, the same spectral characteristics associated with the emergence of the 678 cm⁻¹ peak, most notably the strengthening of the 758 cm⁻¹ peak, were also observed. Serotonin is therefore shown as a reliable marker for Grade 2 biopsies and its detection by Raman spectroscopy may be used to monitor allograft rejection, including cardiac allograft rejection.

### Experimental Example 3

### Immunostaining for Serotonin

To confirm the presence of serotonin in regions of biopsies shown to have signs of transplant rejection (fibrosis) adjacent sections of a heart biopsy were immunostained using an anti-serotonin antibody and stained for collagen, which is characteristic of fibrosis. One section of a Grade 2 heart biopsy was probed for serotonin using a rat monoclonal anti-serotonin antibody (MAb352, Chemicon, Temecula, Calif.) and standard immunohistochemical methodology. An adjacent section was stained using Masson's Trichrom stain (sequential application of Weigert's hematoxylin, Biebrich scarlet and aniline blue stains) using standard immunohistochemical methodology (e.g., Bancroft et al., eds., Theory and Practice of Histological Techniques, 5^{th} edition, Churchill Livingstone, Elsevier Health Sciences, 2002).

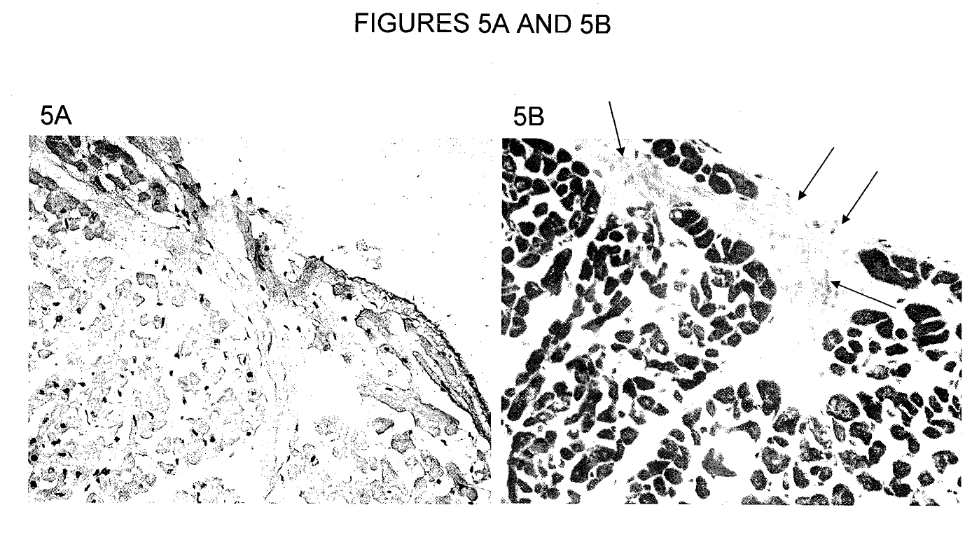

As shown in FIGS. 5A and 5B, the areas that stain positively for collagen also stain positively for serotonin. These data therefore confirm the co-localization of serotonin and collagen and confirmed the detection of serotonin in those regions of Grade 2 biopsies that manifest the excessive production of collagens that leads to cardiac fibrosis.

### Experimental Example 4

### Identification of Abnormal Spectrum by Intensity Ratio

To further assess identification of abnormal levels of serotonin, fifteen endomyocardial biopsies were examined with Raman spectroscopy. Twelve endomyocardial biopsies of Grade 0 and six of Grade 1R were examined. “Grade 1R” refers to the revised grading designation introduced in 2004 (Stewart et al., 2005, J Heart Lung Transplant 24:1710-1720). Grade 1R rejection features interstitial and/or perivascular infiltrate with up to one focus of myocyte damage, and encompasses the “Grade 2” designation of the older system (Stewart et al., ibid).

Raman spectra were obtained at randomly selected positions within the heart biopsy samples without prior knowledge of their rejection grading. The intensity of the obtained Raman spectra was peak-normalized. Between two and seventeen positions were scanned in each biopsy sample.

Abnormal intensity increase was clearly observed with Grade 1R biopsies at 678 cm⁻¹ and 758 cm⁻¹. In addition to the differences seen at 678 cm⁻¹ and 758 cm⁻¹, concomitant strengthening at 824 cm⁻¹ and 938 cm⁻¹ was also observed for the Grade 1R samples.

An intensity ratio of the intensity at the Raman band at 758 cm⁻¹ (I₇₅₈) to the intensity at 718 cm⁻¹ (I₇₁₈) was calculated for each scan. The Raman band at 718 cm⁻¹ was chosen to normalize as it is the closest Raman band to the 758 cm⁻¹ band and is not affected by serotonin in the transplanted graft, or the absence of serotonin in Grade 0 samples. Scans for which the intensity ratio I₇₅₈/I₇₁₈ greater than 1 (I₇₅₈/I₇₁₈>1) were deemed abnormal. For each sample, the percentage of spectra having an abnormal intensity ratio was calculated (% abnormal=100*(Number of spectra with 1758/1718>1 divided by total number of spectra obtained for sample)). These data are summarized in Table 1.

TABLE 1

| Sample | | Number of spectra | Number of spectra | % Ab- |
|---|---|---|---|---|
| number | Grade | with I₇₅₈/I₇₁₈ > 1 | with I₇₅₈/I₇₁₈ ≦ 1 | normal |
| 1 | Grade-0 | 0 | 14 | 0 |
| 2 | | 0 | 9 | 0 |
| 3 | | 0 | 13 | 0 |
| 4 | | 3 | 4 | 43 |
| 5 | | 0 | 6 | 0 |
| 6 | | 0 | 17 | 0 |
| 7 | | 1 | 14 | 6.7 |
| 8 | | 3 | 12 | 20 |
| 9 | | 0 | 15 | 0 |
| 10 | | 0 | 15 | 0 |
| 11 | | 0 | 15 | 0 |
| 12 | | 3 | 12 | 20 |
| | subtotal | 10 | 146 | 6.4 |
| 13 | Grade-1R | 8 | 2 | 80 |
| 14 | | 2 | 0 | 100 |
| 15 | | 4 | 8 | 33.3 |
| 16 | | 6 | 9 | 40 |
| 17 | | 4 | 0 | 100 |
| 18 | | 10 | 6 | 62.5 |
| | subtotal | 34 | 25 | 57.6 |

Of the 156 scans obtained from Grade 0 tissue samples, fewer than 7% (10 scans) had an intensity ratio greater than 1. In contrast, well over half the scans obtained from Grade 1R tissue samples has an intensity ratio greater than 1. Furthermore, all Grade 1K biopsies were observed to have an intensity ratio greater than 1 for at least 33% of the Raman spectra per biopsy. In contrast, only one (Sample 4) out of twelve Grade 0 biopsies was observed to have more than 33% of the Raman spectra obtained demonstrate an abnormal intensity ratio. Therefore, in this study using endomyocardial biopsies (EMB) as the standard of comparison and setting 33% as the decision cut-off point, Raman spectroscopy shows a sensitivity of 100% and a specificity of 92%. In other words, 100% of the Grade 1R biopsies were identified as abnormal, and only 1 of 12 Grade 0 samples were identified as abnormal (i.e., 1 false positive).

Conventional spectral analysis using principle component analysis (PCA) was performed using the 156 baseline spectra from the normal (Grade 0) biopsies and the 34 abnormal spectra from the Grade 1R biopsies. A clear separation between baseline spectra (I₇₅₈/I₇₁₈≦1) and abnorma spectra (I₇₅₈/I₇₁₈>1) was observed. This observation confirms the spectral separation between spectra from normal biopsies and spectra from abnormal biopsies. PCA is contemplated to be useful in developing an automated algorithm for diagnosis in clinical use.

Thus, these data demonstrate that Raman spectroscopy can consistently identify Grade 1R biopsies by detecting an abnormal increase in Raman bands caused by serotonin and further support the value of Raman spectroscopy of serotonin levels as a method for diagnosing organ transplant rejection.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of diagnosing a disease or disorder featuring an abnormal level of a ring-containing molecule in a tissue of a mammal comprising assessing the level of the molecule in the tissue, wherein a change in the level of said molecule in said tissue compared to a reference level of said molecule is indicative of said disease or disorder.

2. The method of claim 1, wherein said ring-containing molecule contains a ring selected from the group consisting of a purine ring, a pyrimidine ring, an indole ring, an imidazole ring and a pyrrolidine ring.

3. The method of claim 1, wherein the ring-containing molecule is selected from the group consisting of adenine, guanine, cytosine, thymidine, uracil, inosine, xanthine, tryptophan, tyrosine, phenylalanine, histidine, serotonin, proline and naturally-occurring derivatives thereof.

4. The method of claim 1, wherein said mammal is a human.

5. A method of diagnosing organ transplant rejection in a mammal, said method comprising assessing the level of serotonin in a transplanted organ or a tissue sample obtained therefrom, wherein an elevated level of serotonin in said transplanted organ or said tissue sample compared to a reference level of serotonin is indicative of transplant rejection.

6. The method of claim 5, wherein said organ transplant comprises an organ selected from the group consisting of heart, heart valves, lung, kidney, liver, cornea, pancreas, heart, intestine, tendons, skin, neural tissues and combinations thereof.

7. The method of claim 5, wherein said organ transplant comprises a heart transplant.

8. The method of claim 5, wherein said organ transplant comprises a kidney transplant.

9. The method of claim 5, wherein assessing the level of serotonin comprises using Raman spectroscopy.

10. The method of claim 9, wherein assessing the level of serotonin comprises using Raman spectroscopy in vivo.

11. The method of claim 9, wherein assessing the level of serotonin comprises assessment of one or more Raman peaks selected from the group consisting of about 678 cm⁻¹, about 758 cm⁻¹, about 820-860 cm⁻¹ and about 938 cm⁻¹.

12. The method of claim 11, wherein assessing the level of serotonin comprises assessment of the about 758 cm⁻¹ Raman peak.

13. The method of claim 9, wherein assessing the level of serotonin comprises obtaining Raman spectra at multiple positions in said transplanted organ or tissue sample obtained therefrom.

14. The method of claim 9, wherein assessing the level of serotonin comprises calculating a ratio of a Raman peak that is indicative of serotonin to a reference Raman peak that is not affected by serotonin.

15. The method of claim 14, wherein the Raman peak that is indicative of serotonin is about 758 cm⁻¹ and the reference Raman peak is about 718 cm⁻¹.

16. The method of claim 5, wherein assessing the level of serotonin comprises an immunoassay.

17. The method of claim 5, wherein said mammal is a human.
